(19) United States
(12) Patent Application Publication
(10) Pub. No.: US 2022/0125688 A1
(43) Pub. Date: Apr. 28, 2022

(54) METHOD FOR ORAL CAVITY CARE

(71) Applicant: Sergey Anatolevich KHOLODOV, Moscow (RU)

(72) Inventor: Sergey Anatolevich KHOLODOV, Moscow (RU)

(21) Appl. No.: 17/423,377

(22) PCT Filed: Jan. 31, 2020
(86) PCT No.: PCT/RU2020/000048

§ 371 Date: Jul. 15, 2021

(30) Foreign Application Priority Data
Feb. 5, 2019 (RU) 2019103229

(51) Int. Cl. A61K 8/04; A61Q 11/00

(52) U.S. Cl. CPC A61K 8/042; A61K 2800/884; A61Q 11/00

(57) ABSTRACT

An oral cavity care method includes daily treatment of the oral cavity with the first and second formulations of REMARS product by alternate exposure, without rinsing the mouth with water between treatments. In this case, depending on the state of the oral cavity, sequential treatment is carried out, first with a restoring solution, to normalize the pH of the oral cavity, and then with a stabilizing solution. The method improves the effectiveness of treatment of oral cavity organs. The advantage of this method consists in the possibility of long-term treatment of the oral cavity at home after the selection of the products by the dentist.

The invention is classified among dentistry, in particular among oral cavity care methods.

The effective cleaning of the oral cavity, in order to ensure the hygiene of the oral cavity organs, requires complex therapeutic and prophylactic care with the combination of two or more active components, the action of which is aimed at the prevention and treatment of different types of pathologies, including control over the formation of dental plaque and calculus deposit. Dental plaque formation is one of the main causes of dental caries, gingivitis, periodontitis, periodontal disease and tooth loss.

An important factor for maintaining the health of the oral cavity is its acid-base balance. The most informative indicator of acid-base equilibrium is the hydrogen-ion exponent (pH value). This indicator varies depending on the area of the oral cavity: acidic pH in the interdental spaces and neutral or slightly alkaline at the tip of the tongue. An integral indicator of acid homeostasis in the oral cavity is the pH of saliva. Normally, the pH of saliva is in the range of 6.8-7.4. The changes in the acid-base balance in the oral cavity can be of two types: acidosis or alkalosis. In case of any changes of homeostasis, it is necessary to distinguish between physiological and pathological changes. Physiological changes are short-term, they do not lead to disruption of normal physiological processes and do not affect the structure and function of the oral cavity tissues. Pathological changes significantly go beyond the boundaries of the norm and lead to structural damage and to dysfunction of certain tissues of the oral cavity: caries, desquamation of the mucous epithelium, deposition of plaque, calculus deposit, periodontitis, periodontal disease.

The salivary secretions, which normally have neutral properties, with an acidity of 6-6.2 causes the destruction of tooth enamel, its demineralization, which subsequently turns into the appearance of carious foci. The gums become inflamed, get swollen, redden, the amount of saliva on the mucous membrane distinctly increases.

The pH value of mixed saliva allows us to estimate the degree of demineralization of hard dental tissues. This biological fluid constantly maintains a neutral acid-base reaction (average value—7.2) of the oral medium due to the proteins and phosphates that it contains. According to the results of modern research, it is the prolonged exposure to acids on the hard tissues of the teeth that provokes the appearance of carious foci. With a decrease in acidity, saliva retains and binds calcium atoms, which leads to demineralization of the teeth. The biological fluid prevents the enamel from dissolving, ensures the diffusion of calcium and phosphorus ions.

A teeth brushing method is also known in the art (RU 2289392, classification A61 [symbol] 8/18, A61 [symbol] 8/64, A61Q11/02, 2005), comprising the treatment of the oral cavity with a two-component dentifrice solution containing an antibacterial compound in combination with an enzyme that provides plaque reduction along with excellent breath freshening characteristics. The two-component dentifrice formulation of the product is effective against plaque and also significantly reduces the level of bacterial species on the surface of the tongue responsible for the production of bad breath. The first component for teeth contains a non-ionic antibacterial compound, and the second component for teeth contains an enzyme. During the dental treatment, the first and second components are combined on the toothbrush and then applied to the surface of the teeth.

However, the known method is not sufficiently effective, due to the fact that the components of the solution are combined on the toothbrush and the interaction of the components of the products occurs first among themselves, thereby entering into a reaction without contact with the biological fluid and without gradual mixing in the oral cavity.

Another method of oral cavity care is also known in the art, comprising the treatment with a solution, containing an abrasive based on fused silica and a source of fluoride. The solution for oral cavity care has a pH value of less than 5.5. This invention is also classified among the methods for increasing fluoride absorption when using an oral solution comprising fluoride and fused silica (RU 2487699, classification A61 [symbol] 8/21, A61 [symbol] 8/22, A61 [symbol] 8/25, A61Q11/00, 2013).

The disadvantage of this method is the lack of control of the oral cavity state. The use of a solution with high acidity for oral cavity treatment is contraindicated in a number of diseases, for example, enamel erosion, enamel hypoplasia, fluorosis, periodontitis, gingivitis, caries in the stain stage. The hydrogen ion concentration of saliva in these diseases (the Ph is less than 6.5) and the use of this method of oral care can lead to multiple caries.

A method of prevention and treatment of initial dental caries is also known in the art (RU 2163114, classification A61 [symbol] 6/00, A61 [symbol] 8/02, 2001), comprising treatment/ mouthwash for 10-15 days with remineralizing solution with redox potential (O-R potential) equal to 700 mV. In this case, an alternate mouthwash of the oral cavity is performed with metastable activated solutions in an amount of 50-100 ml twice a day, before brushing the teeth with anolyte “OXY-DENTA” with a pH of 2.3-3.5 and a positive O-R potential equal to from −800 to −1100 mV, and the concentration of oxidizing agents Cox equal to 10-25 mg/l, and after brushing the teeth with catholyte “DENTA-RED” with a pH of 8.2-9.4 and a negative O-R potential, equal to 300 to 500 mV, while the treatment course is carried out during 30 days 6-7 times a year with intervals of one month between treatment courses.

The disadvantage of this method consists in the complexity of its application. This method can only be used as an addition to basic teeth cleaning. In addition, the known method has a narrowly targeted effect, only the prevention and treatment of caries.

The prototype of the invention is a method of tooth enamel treatment with a two-component composition (RU 2272661, A61Q 11/00, A61K 8/18, 2006), comprising the application of the first solution to the teeth enamel and brushing for 60 seconds, followed by the application of the second solution without rinsing the mouth between brushings. The cleaning is carried out during 30-45 days after breakfast and in the evening before bedtime.

However, this method is narrowly focused and affects only an increase in the time and an improvement of reliability of retention of medicinal components, that restore the integrity of the structure of the teeth enamel, in the pores of the teeth enamel and does not have the ability to impact on the entire oral cavity, because the known method does not take into account the full state of the oral cavity, including the pH of the oral cavity, which depends on many factors, including the presence of fillings, dentures, implants, microorganisms, dental plaque, as well as stomatitis, gingivitis, periodontitis, etc.

The problem of the invention is the development of a method for treatment of the oral cavity, taking into account its physiological state.

The technical result of the invention is to improve the treatment efficiency of oral cavity organs.

The problem raised and the specified technical result are achieved due to the fact that the oral cavity care method includes daily treatment of the oral cavity with the first and second solutions of REMARS product by alternate exposure, without rinsing the mouth with water between treatments According to the invention, depending on the state of the oral cavity, sequential treatment is carried out, first with a restoring solution to normalize the pH of the oral cavity, and then, with a stabilizing solution.

In case of an alkaline pH of the oral cavity, a solution with a pH of 3.0 to 8.0 is used as a restoring agent, and a solution with a pH of 6.0 to 10.0 is used as a stabilizing one.

In case of an acidic pH of the oral cavity, a solution with a pH of 6.0 to 10.0 is used as a restoring agent, and a solution with a pH of 3.0 to 8.0 is used as a stabilizing one.

In case of a neutral pH of the oral cavity, a solution with a pH of 6.8 to 7.2 is used as a restoring agent, and a solution with a pH of 7.3 to 7.8 is used as a stabilizing one and, in this case, the difference between the pH of the restoring and stabilizing solutions is from 0.1 to 7 units.

It is recommended to carry out daily treatment of the oral cavity twice a day with an interval between treatments not more than 20 hours.

The detection of a disease during dental examination of the oral cavity provides a more accurate selection of products for oral cavity hygienic treatment, taking into account the pH level, because each person has his own internal environment pH, depending on his physiological characteristics. Therefore, the selection of hygiene products for oral cavity care should be realized by a dentist.

The sequential oral cavity treatment with two components allows you to preliminarily restore the pH of the oral cavity to its optimal state from 6.8 to 7.4 pH, and then stabilize this state for a significant amount of time until the next oral cavity treatment, thereby providing a neutral pH for a long time, preventing the negative impact of an acidic or alkaline environment on tooth enamel, the appearance of plaque and calculus deposits, and also reduces the risk of microorganisms reproduction and developing of inflammation in the oral cavity.

The difference between the pH of the restoring and stabilizing solutions from 0.1 to 7 units is necessary to ensure the redox reaction of the oral fluid. As a result of the reaction, the mixed saliva shifts to the alkaline or acidic side with gradual subsequent normalization. Moreover, if this difference is less than 0.1 pH, then the redox reaction will not occur, and if the difference exceeds 7 units, then the effect of the products, participating in the reaction, leads to desquamation of the mucosal epithelium, which is a pathological change, leading to structural damage of the oral cavity organs.

As restoring solutions, you can use the following products: REMARSGEL 1 and REMARS JUNIORS 1.

As stabilizing solutions, you can use the following products: REMARSGEL 2 and REMARS JUNIORS 2.

The invention is not limited to the provided description and can be extended within the scope of the claim, for example, other products, that correspond to the declared pH level, can also be used as restoring and stabilizing agents, and various forms can also be used as products.

The advantage of this method is the possibility of long-term oral cavity treatment at home after the selection of products by a dentist. If the doctor's recommendations are followed, the patient, with the help of a restoring and stabilizing solution, will achieve a healthy level of the oral cavity health index.

1. The oral cavity care method, comprising daily oral cavity treatment with the first and second formulations of REMARS product by alternate exposure, without rinsing the mouth with water between treatments, wherein, depending on the state of the oral cavity, sequential treatment is carried out, first with a restoring solution, to normalize the pH of the oral cavity, and then with a stabilizing solution, and in case of an alkaline pH of the oral cavity, a solution with a pH of 3.0 to 8.0 is used as a restoring agent, and a solution with a pH of 6.0 to 10.0 is used as a stabilizing one; in case of an acidic pH of the oral cavity, a solution with a pH of 6.0 to 10.0 is used as a restoring agent, and a solution with a pH of 3.0 to 8.0 is used as a stabilizing one; in case of a neutral pH of the oral cavity, a solution with a pH of 6.8 to 7.2 is used as a restoring agent, and a solution with a pH of 7.3 to 7.8 is used as a stabilizing one, and, in this case, the difference between the pH of the restoring and stabilizing solutions is from 0.1 to 7 units.

2. The method according to clause 1, wherein the daily oral cavity treatment is carried out twice a day with an interval between treatments of not more than 20 hours.

3. (canceled)

4. (canceled)

5. (canceled)

6. (canceled)

7. (canceled)

8. (canceled)

9. (canceled)
